(19) United States
(12) Patent Application Publication
(10) Pub. No.: US 2024/0041025 A1
(43) Pub. Date: Feb. 8, 2024

(54) AN ADHESIVE NANOGEL COMPOSITION AND METHOD OF PREPARATION THEREOF

(71) Applicant: Jyotsnendu GIRI, Hyderabad (IN)

(72) Inventor: Jyotsnendu GIRI, Hyderabad (IN)

(21) Appl. No.: 18/258,930

(22) PCT Filed: Dec. 22, 2021
(86) PCT No.: PCT/IB21/62204

§ 371 Date: Jun. 22, 2023

(30) Foreign Application Priority Data
Dec. 22, 2020 (IN) 202041055925

(51) Int. Cl. A01N 25/04; C09J 133/02; A01N 31/16; A01N 33/12; A01N 47/44; A01P 1/00; C09J 11/06; A61K 8/04; A61K 8/81; A61Q 17/00

(52) U.S. Cl. CPC A01N 25/04; C09J 133/02; A01N 31/16; A01N 33/12; A01N 47/44; A01P 1/00; C09J 11/06; A61K 8/042; A61K 8/8147; A61Q 17/005

(57) ABSTRACT

The present invention relates to an adhesive nanogel composition comprising of bioactive molecules, pre cross-linked polyacrylic acid nanoparticles (pre cross-linked nanoparticles suspension form of carbomer), and pharmaceutically or cosmetically acceptable excipient(s). The present invention also relates to a process for preparation of the adhesive nanogel composition and use of the adhesive nanogel composition as long lasting hand sanitizer or disinfectant of living or non-living surfaces.

### FIELD OF THE INVENTION

The present disclosure generally relates to a pharmaceutical or cosmetic composition. Specifically, the present disclosure relates to an adhesive nanogel composition comprising of bioactive molecules, precross-linked polyacrylic acid nanoparticles (precross-linked nanoparticles suspension form of carbomer), and pharmaceutically or cosmetically acceptable excipient(s). The present disclosure also relates to a process for preparation of the adhesive nanogel composition and use of the adhesive nanogel composition.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Nanogels are submicron-size aqueous dispersions of water-swollen particles, composed of nano-sized three-dimensional highly cross-linked networks of hydrophilic polymers. An active pharmaceutical agent or therapeutic agent with high or low molecular weight can be easily encapsulated into nanogels that can be delivered to the site of action via various routes, including oral, pulmonary, nasal, parenteral and intraocular routes, among others. It has tremendous promising applications in drug delivery and in future nanomedicine. Nanogel-based formulations confirm to be a useful scaffold in nanomedicine including: biosensors, artificial muscles, biomaterials, biochemical separation, cell culture systems, biocatalysis, photonics, biomimetics, drug delivery, anticancer therapy, and the like.

Nanogel networks based on synthetic or natural polymers can be mainly classified into two categories based on their cross-linked structure: chemically cross-linked nanogels which form crosslinking by covalent bonds and physically cross-linked nanogels which form self-assembling through weaker linkages by non-covalent bonds. Crosslinking due to chemical interactions leads to permanent, stable and rigid link in the polymer network. Physical interactions are obtained by polymer chain entanglements or by physical interactions, such as, hydrogen bonds, electrostatic, and vander waals. However, all these methods involve several process steps to get final product of nanogel. In the physical cross-linked method for preparation of nanogel, there is a requirement of nanoreactor templates such as emulsion of water-oil phases followed by the crosslinking of polymers in the nano-reactor space to get nano-sized particles. As an example, polyacrylic acid (PAA) based nanogels have been prepared using a complex inverse-phase microemulsion polymerization using several surfactants and oil-water emulsion templates with multiple steps.

Adhesive nanogel has been of immense interest to the research community as the nanogel particles can adhere on the different surface (living and non-living) for example mucoadhesive nanogel. Mucoadhesive nanogels have attained importance as an alternative to conventional gels in the last decade. Adhesive nanogels are easier to handle and administer as compared to conventional gels due to their lesser viscosity at room temperature.

Currently, the polyacrylic acid based nanogel or other natural or synthetic acid based nanogels are obtained by complex, laborious the multistep preparation process, which severely curtails possibility of cost-effective production of nanogel for different biomedical application.

One of the approach reported in Journal of Materials Chemistry Frontiers, 2(11), September 2018, discloses a antibacterial nanogel where authors used lightly-cross-linked acrylate copolymer nanogel particles of 80 nm (Aqua-SF1, Lubrizol) as starting materials and loaded with chlorhexidine followed by surface functionalization with the cationic polyelectrolyte, poly(diallyldimethylammonium chloride) (PDAC). According to this reference, the authors have demonstrated that the cationic coating of the nanogel strongly amplifies the antimicrobial action of the loaded CHX against both C. reinhardtii, S. cerevisiae, S. aureus and E. coli even for short incubation times. The process of loading antibacterial molecules (chlorhexidine) to prepare antibacterial nanogel composition comprises several steps such as encapsulating chlorhexidine on carbopol nanogel particles by pH change multiple times followed by functionalisation of the CHX-loaded nanogel surface with a cationic polyelectrolyte (PDAC) to obtain the nanogel composition with positive charges. Such thus approach suffers from criticality of pH changes several times, and such method may lead to incomplete functionalization and thereby can affect effectiveness of the nanogel.

Therefore, there exists an unmet need to form the nanogel composition by a simple process such that the nanogel when sprayed or applied on the surface, it adheres on the surface (living or non-living) to form a coating and thereby acting on the desired surface.

One approach in respect of an aqueous cosmetic gel composition is disclosed in US20120263671A1, wherein the composition comprises carbomer, quaternary amine and water. However, the composition does not provide the nanogel as well as cross-linking and thereby can impact the desired adherence.

Another composition in the form of an antimicrobial alcohol-containing composition comprising carbomer, benzalkonium chloride, and other ingredients is disclosed in US6022551A. However, such composition is does not involve cross-linking or provide a nanogel and thereby lack an adherence property for effective activity on the desired surface.

There is, therefore, a need in the art to provide an adhesive nanogel composition suitable for coating on the living or non-living surface and for long lasting delivery of molecule with desired activity. There also remain an unmet need to provide a simple process for preparation of such adhesive nanogel composition.

### OBJECTS OF THE INVENTION

Primary object of the present invention is to provide an adhesive nanogel composition.

Yet another object of the present invention is to provide an adhesive nanogel composition suitable for coating on living or non-living surface and for hand sanitizer or surface disinfectant.

Yet another object of the present invention is to provide an adhesive nanogel composition suitable for encapsulating one or more molecules for their targeted delivery driven by stimuli responsive such as pH and/or temperature induced release of encapsulated molecule.

Another object of the present invention is to provide a one step or simple process for preparation of an adhesive nanogel composition.

Yet another objection of the present invention is to provide a one step or simple process for preparation of an adhesive nanogel composition to encapsulate one or more antibacterial and/or antiviral and/or antifungal into the nanogel system.

Yet another objective of the present invention is to provide one step or simple process for preparation of adhesive nanogel with tailored/variable physicochemical properties such as size and/or charges.

Other objects of the present invention will be apparent from the description of the invention herein below.

### SUMMARY OF THE INVENTION

The present disclosure relates to a composition comprising one or more bioactive molecule(s), pre cross-linked suspension form of carbomer, and pharmaceutically or cosmetically acceptable excipient(s), wherein the pre cross-linked suspension form of carbomer is a suspension of pre cross-linked polyacrylic acid nanoparticles. The pre cross-linked polyacrylic acid nanoparticles present in the composition involve self-assembling of polyacrylic acid with complementary molecules such as quaternary ammonium compounds or ionic/non-ionic surfactants in the aqueous, alcohol, or combination of alcohol and aqueous or other organic solvent mixture and once the liquid phase such as water or alcohol or organic solvent or their mixture evaporated/removed, it adhere on the surface (living or non-living) to form a bioactive molecules loaded nanogel coating.

Accordingly, in one aspect, the present disclosure relates to an adhesive nanogel composition comprising of bioactive molecule(s), pre cross-linked suspension form of carbomer that is pre cross-linked polyacrylic acid nanoparticles, and pharmaceutically or cosmetically acceptable excipient(s).

In one aspect, the present invention relates to an adhesive nanogel composition comprising of:
(a) pre cross-linked polyacrylic acid nanoparticles;
(b) 0% to 95% by weight alcohol;
(c) one or more bioactive molecule(s);
(d) water; and
(e) pharmaceutically or cosmetically acceptable excipient(s).

In another aspect, the present invention relates to an adhesive nanogel composition comprising of:
(a) about 0.1 to 10% by weight of pre cross-linked polyacrylic acid nanoparticles;
(b) 0% to about 95% by weight alcohol;
(c) about 0.1% to about 20% by weight of one or more bioactive molecules;
(d) water; and
(e) about 0.1 to 15% by weight of pharmaceutically or cosmetically acceptable excipient(s).

In another aspect, the present invention relates to an adhesive nanogel composition comprising of:
(a) about 0.1 to 10% by weight of pre cross-linked polyacrylic acid nanoparticles;
(b) about 0.1 to 20% by weight of one or more biomolecule(s) selected from cationic bisbiguanide or cationic molecules;
(c) 0% to 95% by weight alcohol;
(d) about 0.1 to 20% by weight of one or more bioactive molecules; and
(e) about 0.1 to 15% by weight of pharmaceutically or cosmetically acceptable excipient(s).

In certain aspects of the present invention, the pre cross-linked polyacrylic acid nanoparticles are in the form of a pre cross-linked suspension form of carbomer, the same can synthesized or the same can be selected from Carbopol Aqua SF-1, Carbopol Aqua SF-2, Carbopol Aqua SF-1 OS, Novethix L-10, Noverite, Carbopol Aqua 30, Novemer EC-1, and Novemer EC-2.

In another aspect of the present invention, the one or more bioactive molecules is selected from the group consisting of positively charged bioactive molecules, negatively charged bioactive molecules and neutral Zwitter Ionic bioactive molecules.

In another aspect of the present invention, the one or more bioactive molecules are antibacterial/antifungal/antivirus molecule and are selected from benzalkonium chloride, benzethonium chloride, cetalkonium chloride, cetrimide, chlorhexidine, triclosan, povidone iodine, any other bioactive molecules and the like.

In another aspect of the present invention, the one or more bioactive molecules can be molecules such as bisbiguanides, any other bioactive molecules and the like encapsulated into the adhesive nanogel composition.

In yet another aspect of the present invention, the alcohol is selected from the ethanol, isopropyl alcohol, n-propanol, butanol, n-pentanol, hexanol and the like or mixture thereof.

In yet another aspect of the present invention, the pharmaceutically or cosmetically acceptable excipient(s) is selected from emollient, humectant, buffering agent, chelating agent, foam stabilizing agent, fragrances and one or more cationic surfactant.

In another aspect, the present disclosure provides a process for preparation of an adhesive nanogel composition comprising the steps of:
(a) adding pre cross-linked polyacrylic acid nanoparticles suspension into water and stirring to provide a stock solution;
(b) adding the stock solution into alcohol followed by addition of one or more bioactive molecule(s), and pharmaceutically acceptable or cosmetically acceptable excipient(s) and mixing to alcohol to obtain an alcohol based solution;
(c) adjusting the pH of the alcohol based solution in the range of 3.5 to 11 using a base; and
(d) removing the alcohol from the alcohol based solution either spontaneously or under vacuum to obtain adhesive nanogel composition.

In another aspect, the present invention relates to a process for preparation of an adhesive nanogel composition comprising the steps of:
(a) adding pre cross-linked polyacrylic acid nanoparticles suspension into water and stirring to provide a stock solution;
(b) adding one or more bioactive molecule(s), pharmaceutically acceptable or cosmetically acceptable excipient(s) to the stock solution and mixing thoroughly to obtain a homogeneous solution;
(c) adding the homogeneous solution into the alcohol to obtain a alcohol based solution;
(d) adjusting the pH of the alcohol based solution in the range of 3 to 11 using a base; and
(e) removing the alcohol from the alcohol based solution either spontaneously or under vacuum to obtain adhesive nanogel composition.

In another aspect, the present invention relates to a process for preparation of an adhesive aqueous nanogel composition comprising the steps of:
(a) adding one or more bioactive molecules, pre cross-linked polyacrylic acid nanoparticles suspension, and pharmaceutically acceptable or cosmetically acceptable excipient(s) to water to obtain aqueous mixture;
(b) adjusting the pH of the aqueous mixture in the range of 3.5 to 11 using a base to obtain the adhesive nanogel composition.

In yet another aspect of the present invention, the alcohol in the process for preparation of the adhesive nanogel is ethanol, isopropyl alcohol, propanol, n-butanol, n-pentanol and the like or mixture thereof.

In yet another aspect of the present invention, the base used to adjust the pH is sodium hydroxide, triethylamine, or triethanolamine.

In yet another aspect of the present invention, the alcohol in the process for preparation of the adhesive nanogel is present in an amount ranges from about 40% to 95% by weight of the composition.

In yet another aspect of the present invention, the pre cross-linked polyacrylic acid nanoparticles suspension in the stock solution is present in the concentration range of 0.1% to 10% by weight.

In yet another aspect of the present invention, the pre cross-linked suspension form of carbomer in the alcohol solution is present in the concentration range of 0.01% to 5% by weight.

In another aspect, the present invention relates to a hand sanitizer or surface disinfectant nanogel composition comprising of:
(a) one or more bioactive molecules;
(b) pre cross-linked polyacrylic acid nanoparticles suspension;
(c) 60% to 95% by weight ethanol or isopropyl alcohol; and
(d) pharmaceutically or cosmetically acceptable excipient(s).

In another aspect, the present invention relates to a hand sanitizer or surface disinfectant nanogel composition comprising of:
(a) one or more cationic bisbiguanide;
(b) pre cross-linked polyacrylic acid nanoparticles suspension;
(c) pre cross-linked polyacrylic acid nanoparticles suspension;
(d) one or more cationic surfactant;
(e) 60% to 95% by weight ethanol or isopropyl alcohol; and
(f) pharmaceutically or cosmetically acceptable excipient(s).

In yet another aspect, the present invention relates to a hand sanitizer or surface disinfectant nanogel composition comprising of:
(a) about 0.1 to 30% by weight of one or more bioactive molecules;
(b) about 0.1 to 10% by weight of pre cross-linked polyacrylic acid nanoparticles suspension;
(c) optionally 60% to 95% by weight ethanol or isopropyl alcohol; and
(d) about 0.1 to 15% by weight of pharmaceutically or cosmetically acceptable excipient(s).

In yet another aspect, the present invention relates to a surface disinfectant nanogel composition comprising of:
(a) about 0.1 to 20% by weight of one or more bioactive molecules;
(b) about 0.1 to 15% by weight of nanoparticles or metal ions or their complex;
(c) about 0.1 to 10% by weight of pre cross-linked polyacrylic acid nanoparticles suspension;
(d) optionally 60% to 95% by weight ethanol or isopropyl alcohol; and
(e) about 0.1 to 15% by weight of pharmaceutically or cosmetically acceptable excipient(s).

In another aspect, the present invention relates to a hand sanitizer or surface disinfectant nanogel composition comprising of:
(a) one or more cationic bisbiguanide;
(b) pre cross-linked polyacrylic acid nanoparticles suspension;
(c) one or more cationic surfactant;
(d) water; and
(e) pharmaceutically or cosmetically acceptable excipient(s).

In yet another aspect, the present invention relates to a hand sanitizer or surface disinfectant nanogel composition comprising of:
(a) about 0.1 to 30% by weight of one or more bioactive molecules;
(b) about 0.1 to 10% by weight of pre cross-linked polyacrylic acid nanoparticles suspension
(c) about 69% to 99.7% by weight water; and
(d) about 0.1 to 15% by weight of pharmaceutically or cosmetically acceptable excipient(s).

In yet another aspect, the present invention relates to a surface disinfectant nanogel composition comprising of:
(a) about 0.1 to 20% by weight of one or more bioactive molecules;
(b) about 0.1 to 15% by weight of nanoparticles or metal ions or their complex;
(c) about 0.1 to 10% by weight of pre cross-linked polyacrylic acid nanoparticles suspension;
(d) about 69% to 99.7% by weight water; and
(e) about 0.1 to 15% by weight of pharmaceutically or cosmetically acceptable excipient(s).

In certain embodiments, the precross-linked polyacrylic acid nanoparticles suspension can be replaced with a carbopol aqua-SF-1 polymer.

In yet another aspect of the present invention, the hand sanitizer or surface disinfectant composition is in the form of gel, foam or spray.

Various objects, features, aspects and advantages of the inventive subject matter will become more apparent from the following detailed description of preferred embodiments.

### BRIEF DESCRIPTION OF THE FIGURES

FIG. 1A: Shows the nanogel particles (NG-3 and NG-24) are uniformly coated on the Cu grid surface with average particle sizes 150 to 200 nm; FIG. 1B shows Zeta potential and particles size of different nanogel formulations.

FIG. 2(a-b) FE-SEM micrographs of nanoscale coating of adhesive nanogel at different magnifications (Scale bar represents 1 μm and 500 nm respectively). (c) The AFM high profile showing the nanogel coating create the nanoscale roughness on the surface.

FIG. 3 shows pH responsive release active ingredient such as chlorohexidine from the nanogel coated surface.

FIG. 4A Bacterial culture plates of time dependent inhibition of S. aureus by nanogel coated surface. FIG. 4B shows (a) Time dependent killing efficacy of nanogel formulations towards different pathogen with 60 sec of contact time. Long-lasting antimicrobial properties of nanogel coated surfaces, (b) glass and (c) stainless steel surfaces respectively, against different bacteria/fungi monitored for a period of 30 days.

FIG. 5A shows sample collection points from applied hands for Nanogel and control sample and FIG. 5B shows Bacterial culture plates showing the anti-microbial efficacy of nanogel at 4 and 8 h of different volunteers.

FIG. 6A shows sample collection points from applied hands for Nanogel and control sample and FIG. 6B shows Bacterial culture plates showing the anti-microbial efficacy of nanogel at 4 and 8 h of different volunteers.

FIG. 7 Nanogel sample collection points (a) Clinic entrance at Indian Institute of Technology, Hyderabad (Control Surface) (b) The nurse station and (c) Visitor's chair in the clinic where nanogel was applied (d) Buttons outside the lift with control sample application (Control) (e) Lift surfaces where nanogel was applied and buttons inside the lift (inset).

FIG. 8 Representative images of bacterial agar plates used for the field trial (a) Lift surfaces and (b) clinic surfaces sample collection points to evaluate the long-term antimicrobial activity of Nanogel samples. Samples collected from different points are denoted numerically while the control sample-coated surface, labelled C, served as positive control.

FIG. 9 shows images representing contemplated mechanism of action of antimicrobial activity of nanogel coated surface: FIG. 9(a): Step 1: Alcohol droplet on surface instantly kill pathogen instantly; FIG. 9(b): Step 2: Alcohol/liquid evaporate and leaving behind nanogel coating on the surface for long-lasting protection; FIG. 9(c): Nanoscale observation of nanogel coated surface by Atomic Force microscopy; FIG. 9(d): Step 3: Unique positive charges on the nanogel particles, electrostatically attract pathogen (normally negative charges) and kill instantly; and FIG. 9(e): Step 4: Long lasting protection antimicrobial action of nanogel coated surface.

### DETAILED DESCRIPTION OF THE INVENTION

The following is a detailed description of embodiments of the disclosure. The embodiments are in such detail as to clearly communicate the disclosure. However, the amount of detail offered is not intended to limit the anticipated variations of embodiments; on the contrary, the intention is to cover all modifications, equivalents, and alternatives falling within the spirit and scope of the present disclosure as defined by the appended claims.

All publications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Reference throughout this specification to “one embodiment” or “an embodiment” or “another embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

Unless the context requires otherwise, throughout the specification which follow, the word “comprise” and variations thereof, such as, “comprises” and “comprising” are to be construed in an open, inclusive sense that is as “including, but not limited to.”

The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written

The description that follows, and the embodiments described herein, is provided by way of illustration of an example, or examples, of particular embodiments of the principles and aspects of the present disclosure. These examples are provided for the purposes of explanation, and not of limitation, of those principles and of the disclosure.

It should also be appreciated that the present disclosure can be implemented in numerous ways, including as a system, a method or a device. In this specification, these implementations, or any other form that the invention may take, may be referred to as processes. In general, the order of the steps of the disclosed processes may be altered within the scope of the invention.

The headings and abstract of the invention provided herein are for convenience only and do not interpret the scope or meaning of the embodiments.

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

Unless otherwise indicated, the following definitions are set forth to illustrate and define the meaning and scope of the various terms used to describe the invention herein and the appended claims. These definitions should not be interpreted in the literal sense as they are not intended to be general definitions and are relevant only for this application.

As used herein, the terms “composition”or “mixture” are all intended to be used interchangeably.

As used herein, the phrase “pre cross-linked polyacrylic acid nanoparticles” and “pre cross-linked suspension form of carbomer” are used interchangeably.

The present disclosure generally relates to a pharmaceutical or cosmetic composition. Specifically, the present disclosure relates to an adhesive nanogel composition comprising of bioactive molecules, pre cross-linked suspension form of carbomer and pharmaceutically or cosmetically acceptable excipient(s).

The adhesive nanogel composition of the present invention advantageously provides effective coating on any surface including living being skin and nonliving surfaces as well within the liquid phages. The nanogel of the present invention effectively encapsulates the bioactive molecules such as antiseptic, antibacterial, antiviral and any other molecules and releases the encapsulated bioactive molecules on demand/stimuli responsive such as pH and moisture. Further, the nanogel composition of the present invention is suitable for uniformly coating on the surface and killing the pathogen to protect the surface from any contamination. The nanogel composition of the present invention forms layers on the surfaces due to its inherent adhesive nature and release the active molecules as controlled release due to its inherent adhesive nature. The formulation comprising pre cross-linked polyacrylic acid nanoparticles adhesive nanogel remains stable in the aqueous or alcohol-aqueous mixture.

The nanogel composition of the present invention can be formulated and used effectively for sanitization of hand, skin, wound and any surface by choosing appropriate type and quantity of active molecules individually or in combination of antiseptic molecules, such as cetrimide/benzalkonium/benzethonium chloride, chlorhexidine, triclosan, and povidone iodine by applying on the surface by spray or gentle rubbing/covering. The adhesive nanogel can be coated on the surface and release the active molecules on demand when contact with moisture and pH changes/temperature for several hours (more than 8 hours) on living skin and even longer on non-living surface more than 30 days. The adhesive nanogel formulation can be used as long-lasting sanitizer on living skin or non-living surface. In the adhesive nanogel composition, all the ingredients can be in alcohol (ethyl or isopropyl alcohol) and hence, after application the alcohol will work as instant sanitization followed by nanogel layer formation will protect the surface as long-lasting sanitization.

In certain embodiments, the crosslinked polyacrylic acid nanoparticles (CL-PANP) are synthesized.

In certain embodiment, the present disclosure for use in the various adhesive nanogel in accordance with the present disclosure provides preparation of crosslinked polyacrylic acid nanoparticles (CL-PANP) following reported protocols with necessary modification based on the references (Reference 1: Reka Melinda Molnar, Magdolna Bodnar, John F. Hartmann & Janos Borbely. Preparation and characterization of poly(acrylic acid)-based nanoparticles, Colloid Polym Sci (2009) 287:739-744; Reference 2: Marcin Mackiewicz, Zbigniew Stojek and Marcin Karbarz, Synthesis of cross-linked poly(acrylic acid) nanogels in an aqueous environment using precipitation polymerization: unusually high volume change, R. Soc. open sci. 6: 190981, 2019).

In certain embodiments, pre cross-linked polyacrylic acid nanoparticles are synthesized or are pre cross-linked suspension form of carbomer can be selected from Carbopol Aqua SF-1, Carbopol Aqua SF-2, Carbopol Aqua SF-1 OS, Novethix L-10, Noverite, Carbopol Aqua 30, Novemer EC-1, and Novemer EC-2 and other bioactive molecules.

In an embodiment, the present invention relates to an adhesive nanogel composition comprising of:
(a) pre cross-linked polyacrylic acid nanoparticles;
(b) 0% to 95% by weight alcohol;
(c) one or more bioactive molecule(s);
(d) water; and
(e) pharmaceutically or cosmetically acceptable excipient(s).

In an embodiment of the present disclosure provides an adhesive nanogel composition comprises:
(a) about 0.1 to 20% by weight of one or more bioactive molecules;
(b) about 0.1 to 10% by weight of pre cross-linked polyacrylic acid nanoparticles;
(c) 0% to 95% by weight alcohol; and
(d) about 0.1 to 15% by weight of pharmaceutically or cosmetically acceptable excipient(s).

In another embodiment of the present invention, the one or more bioactive molecules is selected from the group consisting of positively charged bioactive molecules, negatively charged bioactive molecules, neutral Zwitter Ionic bioactive molecules and cationic bisbiguanide.

In another embodiment of the present invention, the one or more bioactive molecules are selected from antiseptic molecules, antibacterial molecules, antiallergics molecules, antimycotic molecules, or the like or a combination thereof.

In various embodiments of the present invention, the antibacterial molecules include but not limited to beta lactams, aminoglycosides, macrolides, quinolones and flouroquinolones, Streptogramins, Sulphonamides, tetracyclines, and nitroimidazoles.

In another embodiment of the present invention, the positively charged biomolecules and the negatively charged biomolecules include but not limited to bisbiguanides, benzalkonium chloride, benzethonium chloride, cetalkonium chloride, cetrimide and povidone iodine.

In another embodiment of the present invention, the neutral zwitter ionic biomolecules include but not limited totriclosan.

In another embodiment of the present invention, the one or more bioactive molecules are selected from benzalkonium chloride, benzethonium chloride, cetalkonium chloride, cetrimide, chlorhexidine, triclosan, povidone iodine, any other bioactive molecules and the like.

In another embodiment of the present invention, the one or more cationic bisbiguanide molecules are selected from the group consisting of chlorhexidine, alexidine and octenidine.

In another embodiment of the present invention, the pre cross-linked suspension form of carbomer includes but not limited to Carbopol Aqua SF-1, Carbopol Aqua SF-2, Carbopol Aqua SF-1 OS, Novethix L-10, Noverite, Carbopol Aqua 30, Novemer EC-1, and Novemer EC-2.

In another embodiment of the present invention, the alcohol is selected from ethanol, propanol, isopropyl alcohol, butanol, pentanol, hexanol or mixture thereof. Preferably, the alcohol is ethanol, isopropyl alcohol or mixture thereof.

In an embodiment, the pharmaceutically or cosmetically acceptable excipient(s) is selected from emollients, humectant, buffering agents, chelating agents, foam stabilizing agents, fragrances agents or combination thereof.

In another embodiment of the present invention, the emollients is antioxidant such as vitamin E, caprylic/caprictriglyerides, ceteareth-20, ceteareth-30, cetearyl alcohol, ceteth 20, cetostearyl alcohol, cetyl alcohol, cetylstearyl alcohol, diisopropyladipate, glycerin, glyceryl monooleate, glyceryl monostearate, glyceryl stearate, isopropyl myristate, isopropyl palmitate, white petrolatum, polyethylene glycol, polyoxyethylene glycol fatty alcohol ethers, polyoxypropylene 15-stearyl ether, propylene glycol stearate, squalane, steareth-2 or -100, stearic acid, stearyl alcohol, and the like.

In another embodiment of the present invention, the humectant is butylene glycol, cetyl alcohol, stearyl alcohol, cetearyl alcohol, ethylene glycol, sorbitol, triacetin, glycerol, glyceryl stearate glyceryl oleate, propylene glycol dicaprylate, glyceryl linoleate, glyceryl dibehenate, PEG 120 methyl glucose trioleate and the like.

In another embodiment of the present invention, the foam stabilizing agents is selected from the group consisting of polyethylene oxide, cationic guar gum, cationic cellulose, polyquaternium-2, polyquaternium-4, polyquaternium-6, polyquaternium-7, polyquaternium-10, polyquaternium-11, polyquaternium-15, polyquaternium-16, polyquaternium-22, polyquaternium-42, and combinations thereof and the like.

In another aspect of the present invention, the one or more cationic surfactant is selected from aspartic acid and glutamic acid, lysine, arginine and histidine.

In an embodiment of the present disclosure provides an adhesive alcohol based nanogel composition comprises:
(e) about 0.1 to 20% by weight of one or more bioactive molecules;
(f) about 0.1 to 10% by weight of pre cross-linked polyacrylic acid nanoparticles;
(g) 60% to 95% by weight ethanol or isopropyl alcohol or their mixture; and
(h) about 0.1 to 15% by weight of pharmaceutically or cosmetically acceptable excipient(s).

In another embodiment of the present invention, the nanogel composition comprises:
(a) about 0.1 to 20% by weight of one or more bioactive molecules selected from benzalkonium chloride, benzethonium chloride, cetalkonium chloride, cetrimide, chlorhexidine, triclosan, povidoneiodine, any other bioactive molecules or combination thereof;
(b) about 0.1 to 10% by weight of pre cross-linked polyacrylic acid nanoparticles;
(c) optionally 60% to 95% by weight of alcohol selected from ethanol or isopropyl alcohol, n-propanol or their mixture; and
(d) about 0.1 to 15% by weight of pharmaceutically or cosmetically acceptable excipient(s)selected from emollients, humectant, buffering agents, chelating agents, foam stabilizing agents, fragrances agents or combination thereof.

In an embodiment of the present invention, the nanogel composition comprises:
(a) about 0.1 to 20% by weight of one or more bioactive molecules;
(b) about 0.1 to 10% by weight of pre cross-linked polyacrylic acid nanoparticles;
(c) about 69% to 99.7% by weight water; and
(d) about 0.1 to 15% by weight of pharmaceutically or cosmetically acceptable excipient(s).

In another embodiment of the present invention, the nanogel composition comprises:
(a) about 0.1 to 20% by weight of one or more bioactive molecules selected from benzalkonium chloride, benzethonium chloride, cetalkonium chloride, cetrimide, chlorhexidine, triclosan, povidone iodine, any other bioactive molecules or combination thereof;
(b) about 0.1 to 10% by weight of pre cross-linked polyacrylic acid nanoparticles;
(c) about 69% to 99.7% by weight water; and
(d) about 0.1 to 15% by weight of pharmaceutically or cosmetically acceptable excipient(s)selected from emollients, humectant, buffering agents, chelating agents, foam stabilizing agents, fragrances agents or combination thereof.

In some embodiment of the present invention, the bioactive molecules of the nanogel composition can be used as cross-linking agents or vice-versa.

In some embodiments of the present invention, the bioactive molecules of the nanogel composition can be antiseptic molecules, antibacterial molecules, antiviral molecules, antiallergics molecules, anti-mycotic molecules, or the like.

In various embodiments, the size of the nanogel composition of the present invention is in the range of about 20 nm to 1500 nm. Preferably, the size is in the range of about 20 nm to 400 nm, about 20 nm to 300 nm, about 20 nm to 200 nm, about 20 nm to 100 nm, about 20 nm to 50 nm, about 100 nm to 1500 nm, about 100 nm to 400 nm, about 100 nm to 300 nm, about 100 nm to 200 nm, about 200 nm to 1500 nm, about 200 nm to 400 nm, about 200 nm to 300 nm and about 300 nm to 1500 nm.

In another embodiment of the present invention, the nanogel composition comprises the pre cross-linked polyacrylic acid nanoparticles and quaternary molecules.

In one embodiment of the present invention, the nanogel composition comprises the pre cross-linked polyacrylic acid nanoparticles and quaternary molecules in a molecular ratio range from 1:5 to 1:15. Preferably, in the ratio of 1:1, 1:2, 1:3, 1:4, 1:5, 1:6, 1:7, and 1:8.

In another embodiment of the present invention, the one or more bioactive molecules in the nanogel composition can be present in an amount up to 20% by weight. Preferably, in an amount ranges from about 0.1% to about 15% and about 0.1% to about 10%. More preferably, in an amount of about 0.1%, about 0.5%, about 1%, about 1.5%, about 2%, about 2.5%, about 3%, about 3.5%, about 4%, about 5%, about 6%, about 7%, about 8% about 9%, about 10% and about 15%.

In another embodiment of the present invention, the alcohol in the nanogel formation can be used in an amount up to 95% by weight. Preferably, in an amount of 20%, 30%, 35%, 40%, 45%, 50%, 55%, 60%, 70%, 75%, 80%, and 85%.

In another embodiment of the present invention, the aqueous nanogel formation can comprises water in an amount up to 99.7% by weight. Preferably, in an amount of 65%, 70%, 75%, 80%, and 85%.

In another embodiment of the present invention, the one or more pharmaceutically or cosmetically acceptable excipient(s) in the nanogel composition can be present in an amount up to 10% by weight. Preferably, in an amount of about 0.1%, about 0.5%, about 1%, about 1.5%, about 2%, about 2.5%, about 3%, about 3.5%, about 4%, about 5%, about 6%, about 7%, about 8% about 9%, about 10% and about 15% by weight.

In another embodiment, the adhesive nanogel composition of the present invention has pH responsive properties, the release of encapsulated bioactive molecules is high at pH below 7.4 and continues for longer time i.e., more than 24 hours.

In another embodiment of the present invention, the adhesive nanogel can be coated as uniform layer on the skin, and it can release bioactive molecules on the skin when sweat as the sweat produce water and pH of the sweat normally below 7.4 and up to 5.

In another embodiment of the present invention, the adhesive nanogel can be coated as uniform layer on the skin, and it can release the bioactive molecules on the skin in contact with moisture or in contact with bacteria/virus/fungi thereby it can work as long-lasting disinfectant on the surface.

In another embodiment of the present invention, the hand sanitizer nanogel composition comprises:
(a) one or more bioactive molecules;
(b) pre cross-linked polyacrylic acid nanoparticles;
(c) 0% to 95% by weight ethanol or isopropyl alcohol, n-propanol; and
(d) pharmaceutically or cosmetically acceptable excipient(s).

In yet another embodiment of the present invention, the hand sanitizer nanogel composition comprises:
(a) about 0.1 to 20% by weight of one or more bioactive molecules;
(b) about 0.1 to 10% by weight of pre cross-linked polyacrylic acid nanoparticles;
(c) optionally 0% to 95% by weight ethanol or isopropyl alcohol; and
(d) about 0.1 to 15% by weight of pharmaceutically or cosmetically acceptable excipient(s).

In another embodiment of the present invention, the surface disinfectant nanogel composition comprises:
(a) one or more bioactive molecules;
(b) pre cross-linked polyacrylic acid nanoparticles;
(c) 0% to 90% by weight alcohol; and
(d) pharmaceutically or cosmetically acceptable excipient(s).

In yet another embodiment of the present invention, the surface disinfectant nanogel composition comprises:
(a) about 0.1 to 20% by weight of one or more bioactive molecules;
(b) about 0.1 to 10% by weight of pre cross-linked polyacrylic acid nanoparticles; and
(c) 0% to 95% by weight alcohol;

In another embodiment, the present invention relates to a process for preparation of an adhesive alcohol based nanogel composition comprising the steps of:
(a) adding one or more bioactive molecules, pre cross-linked polyacrylic acid nanoparticles, and pharmaceutically acceptable or cosmetically acceptable excipient(s) to alcohol or organic solvent to obtain an alcohol based mixture;
(b) adjusting the pH of the mixture in the range of 3 to 11 using a base; and
(c) removing the alcohol either spontaneously or under vacuum to obtain a nanogel composition.

In an embodiment of the present invention, the alcohol used in the process of preparation of nanogel composition can be ethanol, propanol, isopropyl alcohol, butanol, pentanol, hexanol or mixture thereof. Preferably, the alcohol is ethanol, propanol, isopropyl alcohol or a mixture thereof.

In some embodiments of the present invention, the alcohol can be replaced with any volatile organic solvents. The volatile organic solvents can be selected from acetone, butanol, dichloromethane, ethylacetate and the like.

In an embodiment of the present invention, the adhesive nanogel composition can be prepared by solvent evaporation method and addition method.

In another embodiment of the present invention, the solvent evaporation method comprises the steps of:
(a) adding pre cross-linked polyacrylic acid nanoparticles polymer into water and stirring to make a stock solution;
(b) adding the stock solution into alcohol followed by addition of one or more bioactive molecules, and pharmaceutically acceptable or cosmetically acceptable excipient(s) to alcohol to obtain alcohol based solution;
(c) adjusting the pH of the alcohol based solution in the range of 3 to 11 using a base; and
(d) removing the alcohol from the alcohol based solution either spontaneously or under vacuum to obtain a nanogel composition.

In yet another embodiment, the addition method comprises the steps of:
(a) preparing pre cross-linked polyacrylic acid nanoparticles suspension in aqueous medium followed by addition of different ingredients, bioactive molecules, emollient, and humectant;
(b) adjusting the pH of the solution to 3 to 11 using a base; and
(c) adding the crosslinker slowly with vigorous stirring to get nanogel;
   wherein the addition can be done vice versa i.e, pre cross-linked polyacrylic acid nanoparticles(with other ingredient) can be added into to crosslinker solution.

In another embodiment, the present invention relates to a process for preparation of an adhesive aqueous nanogel composition comprising the steps of:
(a) adding one or more bioactive molecules, pre cross-linked polyacrylic acid nanoparticles suspension, and pharmaceutically acceptable or cosmetically acceptable excipient(s) to water to obtain aqueous mixture;
(b) adjusting the pH of the aqueous mixture in the range of 3 to 9 using a base to obtain the adhesive nanogel composition.

In another embodiment, the pre cross-linked polyacrylic acid nanoparticles in the stock solution is present in the concentration range of 0.3% to 10%. Preferably, the concentration range of 2% to 6%.

In yet another embodiment, the carbopol aqua-SF polymer in the alcoholic solution is present in the concentration range of 0.01% w/v to 6% w/v. Preferably, in the concentration range of 0.1% w/v to 0.5% w/v, 0.1% w/v to 1% w/v, 0.1% w/v to 1.5% w/v, 0.1% w/v to 2% w/v, 1% w/v to 2% w/v, 2% w/v to 3% w/v, 3% w/v to 4% w/v, and 4% w/v to 6% w/v.

In another embodiment of the present invention, the alcohol used in the process of preparation of the nanogel composition is ethanol, propanol, isopropyl alcohol, butanol, pentanol, hexanol or mixture thereof.

In an embodiment of the present invention, the alcohol in the process of preparation

of the nanogel composition can be present in amount ranges from about 40% to about 95%. Preferably, in an amount of about 40% to about 80%, about 40% to about 70%, about 40% to about 60%, about 50% to about 90%, about 50% to about 80%, about 50% to about 70%, about 50% to about 60%, about 60% to about 90%, about 60% to about 80%, about 60% to about 70%, about 70% to about 90%, about 70% to about 80% and about 80% to about 95%.

In another embodiment of the present invention, the base used in the process of preparation of the nanogel composition for adjusting pH can be any organic or inorganic bases. Preferably, the base is selected from sodium hydroxide (NaOH), triethylaminetriethanolamine or any other chemical bases.

In another embodiment of the present invention, the pH can be adjusted to 3.5 to 11 in the process of preparation of nanogel composition. Preferably, the pH can be adjusted to 3.5 to 8, 3.5 to 7, 3.5 to 6, 3.5 to 5, 4 to 9, 4 to 8, 4 to 7, 4 to 6, 4 to 5, 4 to 7, 5 to 6, 5 to 7, 5 to 8, 5 to 9, 6 to 7, 7 to 8, 7 to 9 and 8 to 9.

None of the present technology prepares the nanogel in one single step and/or instantly on the surface (living or non-living) which open up immense possibility of cost-effective production of nanogel for different biomedical application.

According to the present invention, the adhesive nanogel technology can be formulated as foam, spray, dermal patches, powder form, gel form or liquid form and the like.

The nanogel formulations of the present disclosure are stable in water or mixture of water and volatile solvents for example polar solvents like alcohol. The nanogel formulation of the present invention once applied on the surfaces, liquid component evaporates spontaneously resulting in coating of encapsulated nanogel having active molecules on the surface.

While the foregoing describes various embodiments of the disclosure, other and further embodiments of the disclosure may be devised without departing from the basic scope thereof. The scope of the invention is determined by the claims that follow. The invention is not limited to the described embodiments, versions or examples, which are included to enable a person having ordinary skill in the art to make and use the invention when combined with information and knowledge available to the person having ordinary skill in the art.

### EXAMPLES

The present invention is further explained in the form of following examples. However, it is to be understood that the following examples are merely illustrative and are not to be taken as limitations upon the scope of the invention.

### Example 1: Crosslinked Polyacrylic Acid Nanoparticles (CL-PANP) Preparation

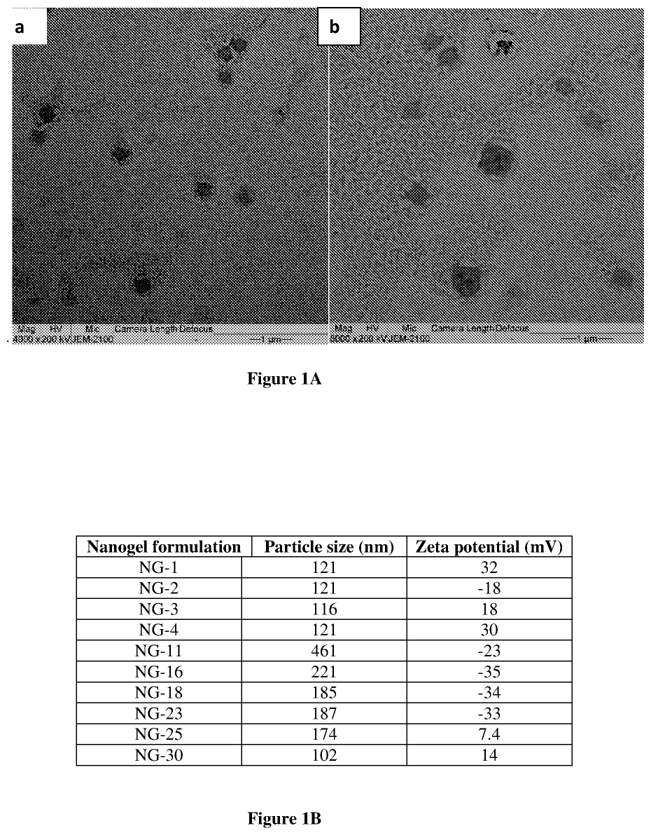

Crosslinked polyacrylic acid nanoparticles (CL-PANP) were prepared following reported protocol with necessary modification (Reference 1: Reka Melinda Molnar, Magdolna Bodnar, John F. Hartmann & Janos Borbely. Preparation and characterization of poly(acrylic acid)-based nanoparticles, Colloid PolymSci (2009) 287:739-744; Reference 2: Marcin Mackiewicz, Zbigniew Stojek and Marcin Karbarz, Synthesis of cross-linked poly(acrylic acid) nanogels in an aqueous environment using precipitation polymerization: unusually high volume change, R. Soc. open sci. 6: 190981, 2019). Polyacrylic acid (PAA) (with molecular weight 50 kD to 800 kD or carbomer or Carbopol) was dissolved in water to make a solution concentration of 1-10 mg/ml, followed by addition of the diamine to the PAA solution and mixed for 30 min at room temperature. 10[3-(dimethylamino)propyl]-3-ethyl-carbodiimide hydrochloride (CDI) was added dropwise and allowed the reaction with stirred at room temperature for 24 h. The solution containing PAA nanoparticles were purified by dialysis for 7 days against distilled water and freeze-dried. Synthesis of cross-linked PAA nanoparticles were prepared with 2,2′-(ethylenedioxy)bis(ethylamine) (EDBEA) at specific stoichiometric cross-linking ratios as described in references (1-2). FIG. 1B shows Zeta potential and particles size of different nanogel formulations.

### Example 2: Adhesive Alcohol Based Nanogel Composition for Inanimate Surface Coating and Sanitizer

Adhesive alcohol based nanogel compositions for inanimate surface coating and sanitizer are provided in below Table 1.

TABLE 1
Compositions for Alcohol based Adhesive nanogel
for inanimate surface coating and sanitizer

| | | NG-1 | NG-2 | NG-3 | NG-4 | NG-5 | NG-6 | NG-7 | NG-8 | NG-9 |
|---|---|---|---|---|---|---|---|---|---|---|
| S. No | Ingredients | Amount % (w/w) | | | | | | | | |
| 1 | Cross-linked | 1.11 | 0.58 | 0.57 | 3.08 | 0.61 | 0.11 | 0.5 | 0.68 | 0.68 |
| | PAA-NP | | | | | | | | | |
| 2 | Ethanol | 61.22 | 64.22 | 0 | 56.80 | 92 | 0 | 39.45 | 0 | 62.76 |
| 3 | Iso Propyl | 0 | 0 | 63.68 | 0 | 0 | 64.4 | 0 | 63.12 | 0 |
| | Alcohol | | | | | | | | | |
| 4 | Benzalkonium | 4.43 | 0.23 | 1.15 | 9.25 | 1.22 | 0.2 | 0.2 | 1.14 | 1.13 |
| | Chloride | | | | | | | | | |
| 5 | Chlorohexidine | 0 | 0 | 0 | 0 | 0 | 0 | 0 | 0.8 | 0 |
| 6 | Triclosan | 0 | 0 | 0 | 0 | 0 | 0 | 0 | 0 | 1.30 |
| 7 | Water | 33.24 | 34.88 | 34.50 | 30.85 | 6.13 | 35.5 | 60 | 34.28 | 34.28 |

Cross-linked PAA-NP: Cross-linked polyacrylic acid nanoparticles

For each composition, as per the concentrations in above Table 1, components were taken for preparing an alcohol based adhesive nanogel. A stock solution was prepared by mixing cross-linked polyacrylic acid nanoparticles prepared as per Example 1 was taken and ethanol or isopropyl alcohol was added into it. After, proper mixing, benzalkonium chloride, Chlorohexidine, and triclosan were added under stirring. The required quantity of water was added and pH was maintained to 7.0 using NaOH. The obtained alcohol based solution is suitable for surface coating.

### Examples 3:Compositions for Aqueous Adhesive Nanogel for Inanimate Surface Coating and Sanitizer

Compositions for aqueous adhesive nanogel inanimate surface coating and sanitizer are provided in below Table 2.

TABLE 2
Compositions for Aqueous Adhesive nanogel for inanimate surface coating and sanitizer

| | | NG-10 | NG-11 | NG-12 | NG-13 | NG-14 | NG-15 | NG-16 |
|---|---|---|---|---|---|---|---|---|
| S. No | Ingredients | Amount % (w/w) | | | | | | |
| 1 | Cross-linked PAA-NP | 0.47 | 0.49 | 0.49 | 2.67 | 0.49 | 0.47 | 0.49 |
| 3 | Benzalkonium Chloride | 3.82 | 0.19 | 0.99 | 8.03 | 0.99 | 3.82 | 0.19 |
| 5 | Water | 95.70 | 99.30 | 99.00 | 89.28 | 99.0 | 95.6 | 99.30 |

Cross-linked PAA-NP: Cross-linked polyacrylic acid nanoparticles

### Example 4: An Adhesive Alcohol Based Nanogel for Long-Lasting Animate Surface Sanitizer

Compositions for adhesive alcohol based nanogel for Long-Lasting animate surface sanitizer are provided in below Table 3.

TABLE 3
Compositions for adhesive alcohol based nanogel
for Long-Lasting animate surface sanitizer

| | | NG- | NG- | NG- | NG- | NG- | NG- | NG- | NG- |
|---|---|---|---|---|---|---|---|---|---|
| S. | | 17 | 18 | 19 | 20 | 21 | 22 | 23 | 24 |
| No | Ingredients | Amount % (w/w) | | | | | | | |
| 1 | Cross-linked | 1.10 | 1.09 | 1.04 | 0.34 | 1.05 | 1.16 | 0.79 | 1.10 |
| | PAA-NP | | | | | | | | |
| 2 | Ethanol | 0 | 60.20 | 0 | 62.95 | 0 | 0 | 62 | 0 |
| 3 | Iso Propyl | 60.87 | 0 | 57.69 | 0 | 41.54 | 87.71 | 0 | 60.87 |
| | Alcohol | | | | | | | | |
| 4 | Cetrimide | 1.13 | 2.20 | 3.27 | 4.17 | 1.02 | 2.10 | 2.33 | 1.13 |
| 5 | Chlorohexidine | 0.3 | 2.24 | 2.18 | 5.22 | 0.91 | 2.10 | 2.33 | 0.34 |
| 6 | Triclosan | 0 | 0 | 0 | 0 | 0 | 0 | 0 | 1 |
| 7 | Glycerol | 0.50 | 0.55 | 0.54 | 0.52 | 0.56 | 0.52 | 0.58 | 0.50 |
| 8 | Water | 34.1 | 33.06 | 32.70 | 31.33 | 34.19 | 52.65 | 5.84 | 33 |

Cross-linked PAA-NP: Cross-linked polyacrylic acid nanoparticles

For each composition, as per the concentrations in above Table 3, components were taken for preparing an alcohol based adhesive nanogel. A stock solution was prepared by mixing cross-linked polyacrylic acid nanoparticles prepared as per Example 1 with ethanol or isopropyl alcohol. Cetrimide, chlorhexidine gluconate, triclosan, were was added under stirring to obtain alcohol based adhesive gel solution. The pH of the alcohol based gel solution containing different ingredients wad adjusted to 7.4 using triethanolamine to obtain the nanogel composition suitable as hand sanitizer.

### Example 5: Compositions for Aqueous Adhesive Nanogel for Long-Lasting Animate Surface Sanitizer

Compositions for aqueous adhesive nanogel for Long-Lasting animate surface sanitizer are provided in below Table 4.

TABLE 4
Compositions for Aqueous Adhesive nanogel for animate surface coating and sanitizer

| | | NG-25 | NG-26 | NG-27 | NG-28 | NG-29 | NG-30 |
|---|---|---|---|---|---|---|---|
| Sr. No | Ingredient | Amount % (w/w) | | | | | |
| 1 | Cross-linked PAA-NP | 0.95 | 0.94 | 0.90 | 0.1 | 0.58 | 1.07 |
| 2 | Cetrimide | 1.90 | 2.83 | 3.63 | 0.3 | 1.45 | 2.03 |
| 3 | Chlorhexidine gluconate | 1.90 | 1.88 | 4.54 | 0.4 | 0.83 | 1.06 |
| 4 | Water | 95.23 | 94.33 | 90.90 | 100 | 97.08 | 96.15 |

Cross-linked PAA-NP: Cross-linked polyacrylic acid nanoparticles

Cross-linked polyacrylic acid nanoparticles suspension in water was prepared and the pH of the suspension was adjusted to 7.0 using triethanolamine. To the suspension, the cetrimide and chlorhexidine gluconate were added under stirring and the pH was adjusted to 7.4 using triethanolamine to obtain water based adhesive nanogel for long-lasting hand sanitizer.

### Example 6

### I. Characterization of Adhesive Nanogels

The particles size and zeta potential of the nanoparticles of nanogels NG-3 and NG-24 were measured using DLS (Zetasizer, NanoZS, UK). The dispersed nanoparticles were diluted with adequate amount of ultrapure water (˜3 mg/ml). Subsequently, it was filled into disposable zeta cell and size as well as zeta potential was measured. Similarly, nanogel particles size was observed under Transmission Microscope, where nanogel formulations was drop casted on TEM Cu grid. As can be seen from the nanogel particles (NG-3 and NG-24) are uniformly coated on the Cu grid surface with average particle sizes 150 to 200 nm.

### II. Microscopic Observation of Nanogel Coated Surface

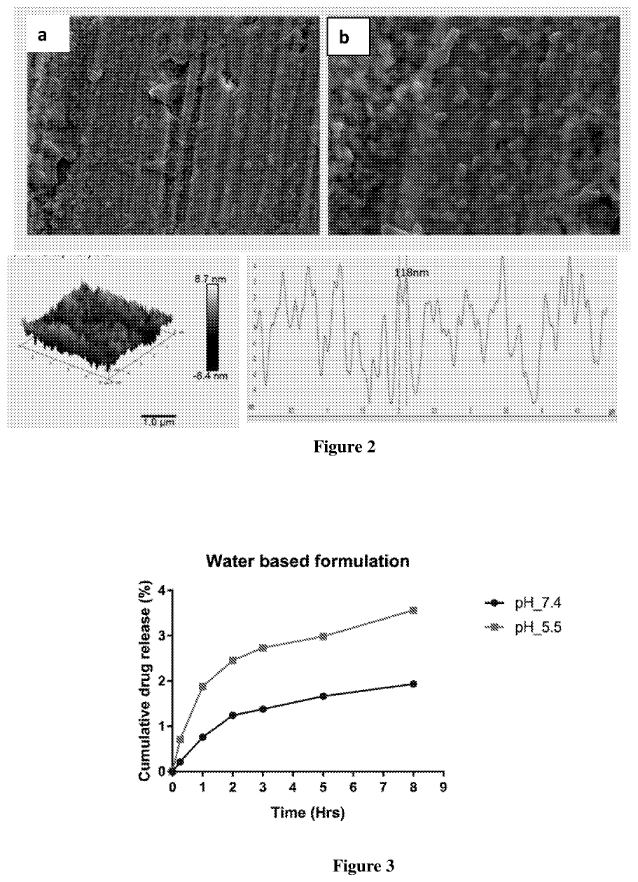

The particles size, morphology of obtained nanoparticles and surface topography and coating behavior of nanoformulation were studied using Atomic Force Microscopy (Veeco-Bruker) and Field emission electron microscope (Joel, Japan). For FESEM analysis one drop of nanoformulation was placed on the alumina stub and allowed to dry for 10-15 minutes. The formed film was observed under the FESEM. The macroscopic formed coating was removed scratched from one part of surface and the scratched region was also observed under FESEM microscope to check presence of nano scale coating of nanogel formulation. AFM height profile showing the presence of nanotopography or nano-roughness of nanogel coated surface. As can be seen from FIG. 2. (a-b) shows FE-SEM micrographs of nanoscale coating of adhesive nanogel at different magnifications (Scale bar represents 1 μm and 500 nm respectively). (c) The AFM high profile shows the nanogel coating created the nanoscale roughness on the surface.

### Example 7: Release Profile of Active Ingredient from the Nanogel Coated Surface

Release profiles of different nanogel samples (NG-29) were performed where 2 ml of formulation was sprayed in a 60 mm glass disc and dried under vacuum at room temperature (25° C.). After drying, 8 ml of PBS having respective pH value (pH 7.4 and pH 5.5) was added on the coated sample. At predetermined time point 1 ml release medium was collected and volume was mentioned with fresh PBS. The concentration of chlorhexidine was determined in HPLC. As cab be seen from FIG. 3, pH responsive release active ingredient such as chlorohexidine was from the nanogel coated surface for a longer duration over 8 hours.

### Example 8: Time-Kill Effect: (Determination of Minimum Time to Kill the Bacteria)

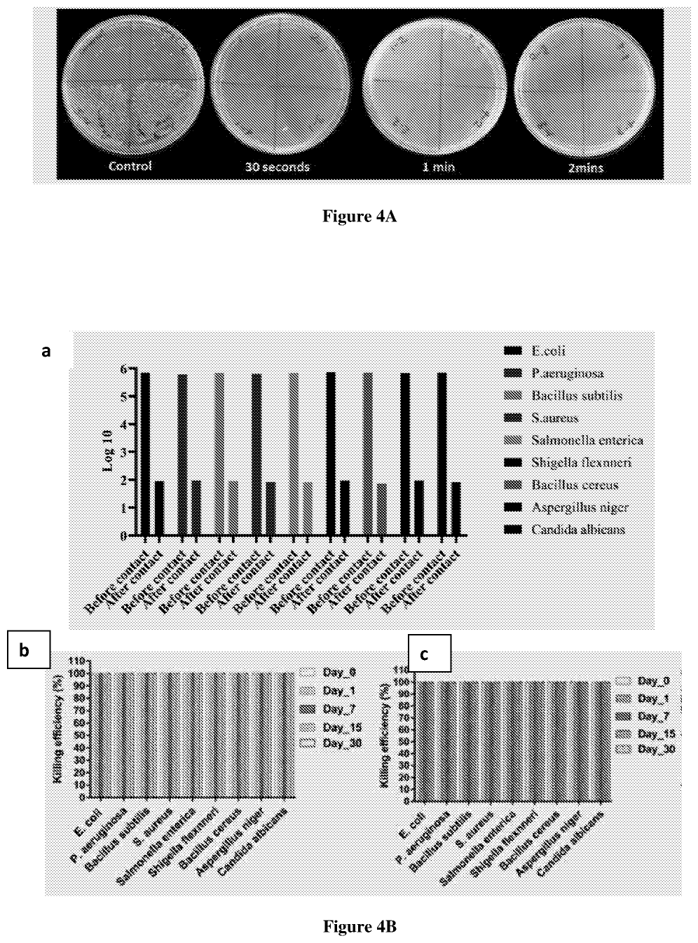

To determine the appropriate length of time needed to inactivate the microbes by nanogels, the time-kill assay was performed on a glass surface. Standard laboratory microcopic slides with 75 mm by 25 mm size were taken and dipped completely in ethanol and let it to dry on tissue paper for 5 mins. 0.1 ml of nanogel sample NG-3 was applied on glass slides in triplicates covering 70% of the slide area and were let it to dry for 30 mins inside a microbiological hood. Freshly grown both E. coli and S. aureus bacterial cells at cells in 0.1 ml of suspension were applied on the area of formulation quickly and incubated for 15 sec, 30 sec, 1 min and 5 mins. After the appropriate time of incubation, the slides were immediately transferred to 30 ml of LB broth media in 50 ml falcon. Falcon containing the slide were sonicated in a bath sonicator for 20 secs and incubated at 37° C. incubator for 5 mins with 200 rpm shaking. 20 ul of samples were streaked on LB agar plates and incubated at 37° C. dry incubator O/N. Next day the presence of recovered colony forming units (CFUs) of bacterial cells were observed and counted. FIG. 4 shows representative bacterial culture plates of time dependent inhibition of S. aureus by nanogel coated surface

### Example 9: Antimicrobial Efficacy of Formulation on Various Surfaces at Various Time Points Against Various Microorganism

Antibacterial efficacy of nano-coating formed by nanogel formulation NG-5 on two different surfaces ex., glass and stainless steel at various time points of 0, 7, 10, 15 and 30 days after incubating for 30 and 60 sec. were evaluated using various pathogens. Glass/stainless steel slides were dipped completely in ethanol and let it to dry on tissue paper for 5 mins. 100 ul of sanitizer nanogel samples applied on glass slides in triplicates covering 60-70% of the slide area and were let it to dry for 30 mins inside a microbiological hood. Freshly grown range of bacterial and fungus cells at 5×10⁴ cells in 100 ul of suspension were applied on the area of samples and incubated for 30 sec and 1 min. Slides were immediately transferred to 30 ml of LB broth media in 50 ml falcon. Falcon containing the slide were sonicated in bath sonicator for 20 secs and incubated at 37° C. incubator for 5 mins with 200 rpm shaking. 20 ul samples plated on agar plates and incubated at 37° C. dry incubator O/N and next day the presence of colonies were observed. Slides were stored for 30 days and antimicrobial efficacy of the surface was determined at specific time interval. FIG. 4 (a) shows time dependent killing efficacy of nanogel formulation NG-5 towards different pathogen with 60 sec of contact time. FIG. 4 shows the long-lasting antimicrobial properties of nanogel coated surfaces, (b) shows glass and (c) shows stainless steel surfaces respectively, against different bacteria/fungi monitored for a period of 30 days.

### Example 10: Determination of Long-Lasting Antibacterial Activity on Different Surfaces

To determine the long-lasting antimicrobial efficacy of aqueous adhesive nanogel on animate surface and sanitizer following method was followed:

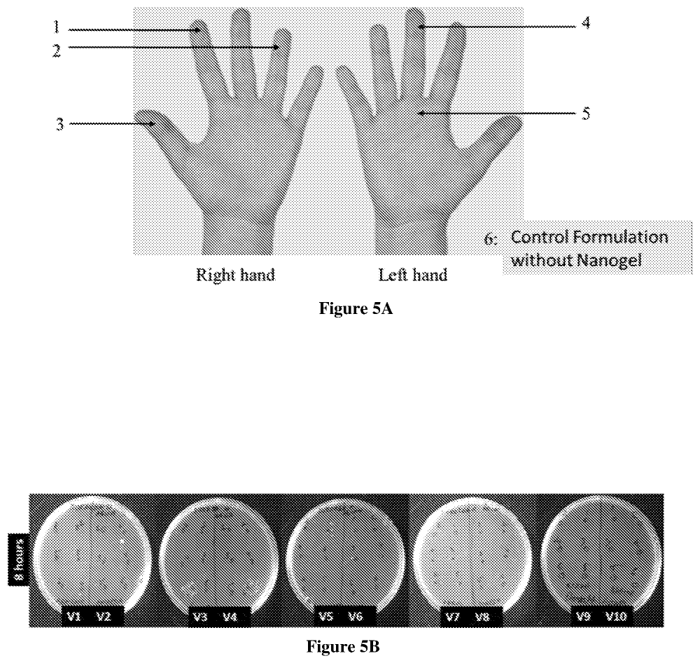

Method: Four puffs of Nanogel formulation (NG-29) and control formulation, the aqueous sanitizer was applied on the hands of the volunteers. The sanitizer was properly rubbed on the hands and in between fingers until dried. Similar to nanogel, the volunteers of nanogel were divided into two groups of 4 h and 8 h. The volunteers were asked to do their regular work but not to wash their hands off. Samples were collected at the respective time points using sterile swabs and were dabbed on LB agar plates. The plates were incubated for 12-16 h and then checked for microbial growth. Control formulation having similar quantity of active ingredient of NG-29, i.e., cetrimide and chlorohexidine without polymers nanoparticles. FIGS. 5 shows bacterial culture plates showing the anti-microbial efficacy of nanogel at 4 and 8 h of different volunteers.

Observation: The efficacy of the aqueous adhesive nanogel foam sanitizer was demonstrated on the volunteers (8-10) for 4 and 8 h. 90-95% killing of microbes were observed after spraying nanogel as observed by the absence of microbial colonies on the LB agar plates.

### Example 11: Determination of the Long-Lasting Antimicrobial Efficacy of Alcohol Based Adhesive Nanogel on Animate Surface and Sanitizer

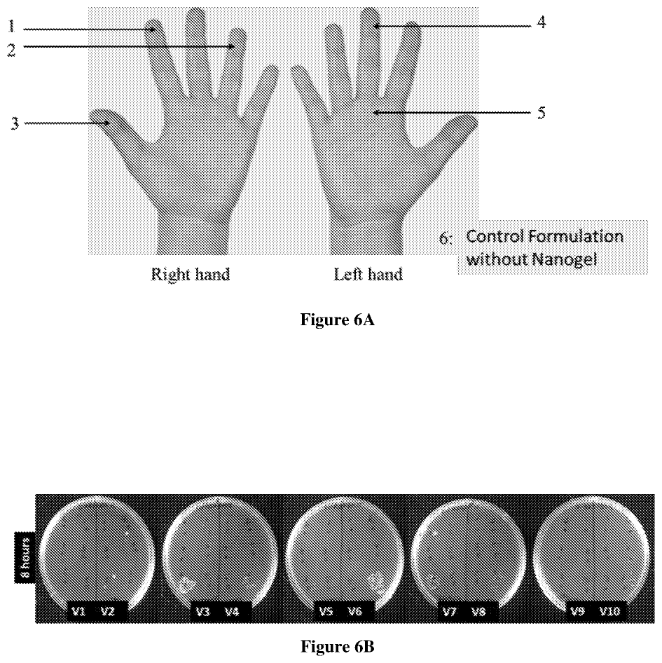

Method: Application Method: nanogel formulation (NG-17), and control formulation the alcohol based sanitizer, was sprayed on both sides of the hands of volunteers and was allowed to dry without rubbing. The volunteers were divided in two groups of 4 h and 8 h, based on the time point at which the samples were collected. The volunteers were allowed to do their regular work but were asked not to wash their hands. After the respective time points, the samples were collected from their hands using sterile swabs. The swabs were dabbed on LB agar plates and incubated for 12-16 h. The plates were then checked for microbial growth. Control formulation having similar quantity of active ingredient of NG-17, i.e., cetrimide and chlorohexidine without polymers nanoparticles. FIG. 6 shows bacterial culture plates showing the anti-microbial efficacy of nanogel and control samples at 4 and 8 h of different volunteers.

Observation: The alcohol based sanitizer was sprayed on the hands of the volunteers and samples collected after 4 and 8 h. The absence of microbial growth (no colonies) from swabs dabbed from nanogel and control samples applied hands shows that nanogel completely subdued the growth of microbes. As observed from the colonies in control formulation applied hands, the nanogel applied hand surfaces of the volunteers were completely free of microbes.

### Example 12: Determination of the Long-Lasting Antimicrobial Efficacy of Adhesive Nanogel on Inanimate Surface

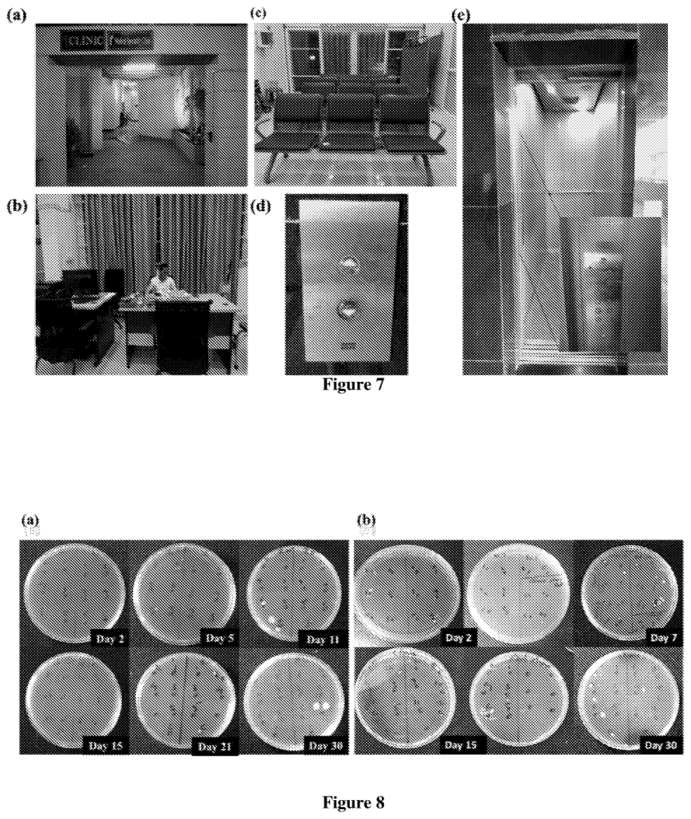

Method: The antimicrobial activity of nanogel formulation NG-5 was assessed on frequently used surfaces such as lift, door handles and clinic etc. for a period of 35 days. Respective surfaces were clean from dust and nanogel formulation and control formulation applied. Nanogel and control formulation was sprayed different surface using standard spray machines producing fine mist and were allowed to complete dry. Till the entire duration of experiment, no other decontaminators were used on those surfaces. For the lift, samples were collected from the lift buttons, sides and the handle. Button outside the lift served as control where nothing was sprayed. For the clinic, samples were collected from the nurse station (chair handle and table) and visitors' chair. The entrance door handle served as the control with spray of control formulation having BKC without polymer nanoparticles. The samples from respective places were collected using sterile swabs and were then dabbed on Luria Bertani (LB) agar plates. The plates were incubated in 37° C. for 12-16 h. Microbial colony formation was observed after the incubation time. FIG. 7 shows nanogel sample collection points (a) Clinic entrance at Indian Institute of Technology, Hyderabad (Control Surface) (b) The nurse station and (c) Visitor's chair in the clinic where nanogel was applied (d) Buttons outside the lift with control sample application (Control) (e) Lift surfaces where nanogel was applied and buttons inside the lift (inset). FIG. 8 shows representative images of bacterial agar plates used for the field trial (a) Lift surfaces and (b) clinic surfaces sample collection points to evaluate the long-term antimicrobial activity of Nanogel samples. Samples collected from different points are denoted numerically while the control sample-coated surface, labelled C, served as positive control.

Observation: Nanogel coated surface proves to be a strong disinfectant as minimal growth of microbes was observed in both lift and clinic even after 30 days of spraying Nanogel disinfectant. The long-lasting efficacy of the surface disinfectant is demonstrated by the inhibition of microbial colonies on the agar plates from samples collected over the same period. The nanogel formulation coating provided robust antibacterial efficacy in our field trial.

### Example 13: In Vitro Anti-Viral Property (Anti-COVID Test) (Plaque Assay)

The anti-viral test for different nanogel formulations (NG-5, NG-18, NG-26) was performed at THSTI, Faridabad. 70111 of nano-formulation was applied on sterile glass slide and kept overnight for drying. Next day, 5×106 PFU/ml SARS-CoV-2 virus was added on the coated zone on the glass slide and incubated for 1 minute and 2.5 minutes. Sterile glass slide with no coating served as positive control while adding uninfected cells on coated glass slide served as negative control. After incubation, treated virus was collected and serially diluted to estimate the virus titer by plaque assay in VeroE6 cells monolayer as per the SOP (THSTI/TEC/37). Plaques were counted manually and reported for at least 10 plaques for counting in appropriate dilution in test samples and positive control. No plaques in negative control. Results are reported in Table 5:

TABLE 5

| S No | Sample Details | Time | | Average titre (n = 3) PFU/ml |
|---|---|---|---|---|
| 1 | Control | 1 | min | 3.73E+06 |
| 2 | | 2.5 | min | 5.20E+06 |
| 3 | NG-5 | 1 | min | 0.00E+00 |
| 4 | | 2.5 | min | 0.00E+00 |
| 5 | NG-18 | 1 | min | 0.00E+00 |
| 6 | | 2.5 | min | 0.00E+00 |
| 7 | NG-26 | 1 | min | 6.03E+06 |
| 8 | | 2.5 | min | 5.48E+05 |

Observation: Nanogel formulation NG-5 and NG-18 showed complete inhibition of SARS-CoV-2, at 1 min and 2.5 min. NG-26 did not show any inhibition of SARS-CoV-2 with 1 min incubation, however by 2.5 min incubation resulted 90% of reduction of virus titers.

### Example 14: Mechanism of Action of Antimicrobial Activity of Nanogel Coated Surface

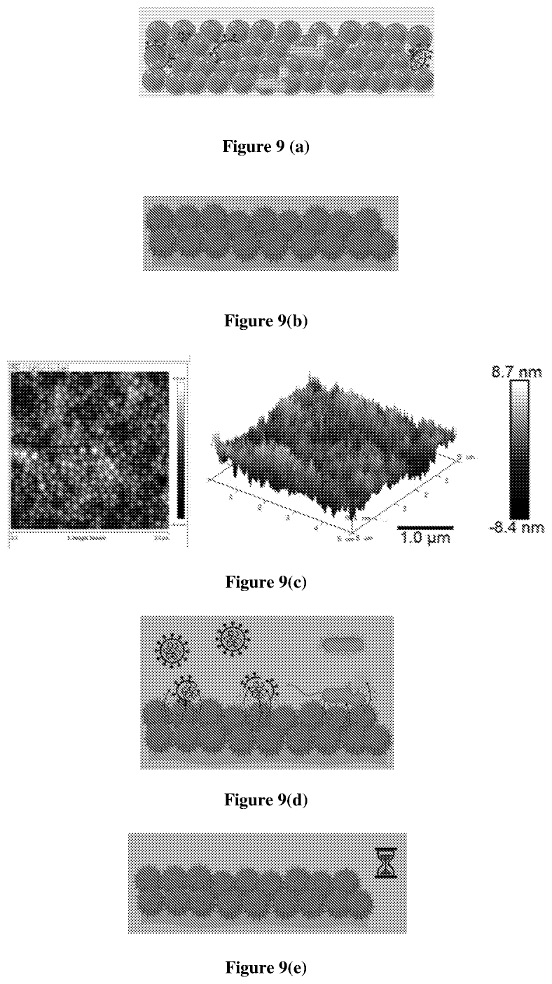

Without bound by any theory, mechanism of action of antimicrobial activity of nanogel coated surface is represented in FIG. 9:
Step 1: Droplets of alcohol based nanogel for example NG-3 or NG-5 when applied on surface, instantly kills pathogen instantly (FIG. 9(a)).
Step 2: Alcohol/liquid evaporate and leaving behind nanogel coating on the surface for long-lasting protection (FIG. 9(b)). Nanoscale observation of nanogel coated surface by Atomic Force microscopy is represented in FIG. 9(c).
Step 3: Unique positive charges on the nanogel particles, electrostatically attract pathogen (normally negative charges) and kill instantly (<60 sec) by synergistic action of physical disruption by nano-topography, molecular needles, and chemically lysis from released molecules close vicinity of pathogen (FIG. 9(d)).
Step 4: Long lasting protection antimicrobial action of nanogel coated surface (FIG. 9(e)).

### ADVANTAGES OF THE PRESENT INVENTION

The main advantage of the present invention is simple cost effective method to prepare bioactive molecules loaded nanogel with tailored physicochemical properties such as size and zeta potential for different long lasting applications.

The adhesive nanogel formulation of the present invention advantageously shows stimuli response properties (pH-responsive) such as release of bioactive molecules.

The adhesive nanogel formulation of the present invention is useful in coating on the living or nonliving surface.

The adhesive nanogel formulation of the present invention shows long lasting Hand Sanitizer or Surface Disinfectant activity.

The adhesive nanogel formulation of the present invention releases the encapsulated bioactive molecules highly at pH below 7.4 (mild acidic) and releases for long time i.e. more than 24 hours.

The adhesive nanogel formulation of the present invention can coat on the uniform layer on the skin, and it can release bioactive molecules on the skin when sweat as the sweat produce water and pH of sweat normally below 7.4, and up to 5.

The nanogel formulation of the present invention can coat on the surface and hold bioactive molecules, and release the molecules in contact of moisture or when in contact with bacteria/virus/fungi, thus can work as Long-Lasting disinfectant on the surface.

The present disclosure provides a long-lasting antimicrobial adhesive nanogel comprising composition in accordance with the present invention for inanimate or animate surface.

1. An adhesive nanogel composition comprises:
(a) about 0.1 to 20% by weight of one or more bioactive molecules;
(b) about 0.1 to 10% by weight of pre cross-linked polyacrylic acid nanoparticles;
(c) 0% to 95% by weight alcohol; and
(d) about 0.1 to 15% by weight of pharmaceutically or cosmetically acceptable excipient(s).

2. The adhesive nanogel composition as claimed in claim 1, wherein the one or more bioactive molecules is selected from the group consisting of positively charged bioactive molecules, negatively charged bioactive molecules, neutral Zwitter Ionic bioactive molecules, and cationic bisbiguanide.

3. The adhesive nanogel composition as claimed in claim 1, wherein the one or more bioactive molecules are selected from antiseptic molecules, antibacterial molecules, antiallergics molecules, antimycotic molecules, or the like or a combination thereof.

4. The adhesive nanogel composition as claimed in claim 3, wherein the antibacterial molecules include but not limited to beta lactams, aminoglycosides, macrolides, quinolones and flouroquinolones, Streptogramins, Sulphonamides, tetracyclines, and nitroimidazoles.

5. The adhesive nanogel composition as claimed in claim 2, wherein the positively charged biomolecules and the negatively charged biomolecules include but not limited to bisbiguanides, benzalkonium chloride, benzethonium chloride, cetalkonium chloride, cetrimide and povidone iodine.

6. The adhesive nanogel composition as claimed in claim 2, wherein the neutral zwitter ionic biomolecules is triclosan.

7. The adhesive nanogel composition as claimed in claim 2, wherein the one or more cationic bisbiguanide molecules are selected from the group consisting of chlorhexidine, alexidine and octenidine.

8. The adhesive nanogel composition as claimed in claim 1, wherein the one or more bioactive molecules are selected from benzalkonium chloride, benzethonium chloride, cetalkonium chloride, cetrimide, chlorhexidine, triclosan, and povidone iodine.

9. The adhesive nanogel composition as claimed in claim 1, wherein the pre cross-linked polyacrylic acid nanoparticles is synthesized.

10. The adhesive nanogel composition as claimed in claim 1, wherein the pre cross-linked polyacrylic acid nanoparticles is a pre cross-linked suspension form of carbomer selected from Carbopol Aqua SF-1, Carbopol Aqua SF-2, Carbopol Aqua SF-1 OS, Novethix L-10, Noverite, Carbopol Aqua 30, Novemer EC-1, and Novemer EC-2.

11. The adhesive nanogel composition as claimed in claim 1, wherein the alcohol is selected from ethanol, propanol, isopropyl alcohol, butanol, pentanol, hexanol or mixture thereof.

12. The adhesive nanogel composition as claimed in claim 1, wherein the alcohol is present in the range of 60% to 95%.

13. The adhesive nanogel composition as claimed in claim 1, wherein the pharmaceutically or cosmetically acceptable excipient(s) is selected from emollients, humectant, buffering agents, chelating agents, foam stabilizing agents, fragrances agents or combination thereof.

14. The adhesive nanogel composition as claimed m claim 1, wherein the adhesive nanogel composition is pH responsive and releases encapsulated bioactive molecules at pH below 7.4.

15. A long-lasting antimicrobial adhesive nanogel comprising composition as claimed in claim 1 for inanimate or animate surface.

16. A process for preparation of an adhesive alcohol based nanogel composition comprising:
(a) adding one or more bioactive molecules, pre cross-linked polyacrylic acid nanoparticles, and pharmaceutically acceptable or cosmetically acceptable excipient(s) to alcohol to obtain an alcohol based mixture; and
(b) adjusting the pH of the mixture in the range of 3 to 11 using a base.

17. A process for preparation of an adhesive aqueous nanogel composition comprising:
(a) adding one or more bioactive molecules, pre cross-linked polyacrylic acid nanoparticles suspension, and pharmaceutically acceptable or cosmetically acceptable excipient(s) to water to obtain aqueous mixture;
(b) adjusting the pH of the aqueous mixture in the range of 3 to 11 using a base to obtain the adhesive nanogel composition.

18. The process as claimed in claim 16, wherein the pre cross-linked polyacrylic acid nanoparticles in present in the concentration range of 0.3% to 10%, preferably in the concentration range of 2% to 6%.

19. The process as claimed in claim 17, wherein the pre cross-linked polyacrylic acid nanoparticles in present in the concentration range of 0.3% to 10%, preferably in the concentration range of 2% to 6%.
